(19) United States
(12) Patent Application Publication
(10) Pub. No.: US 2007/0098664 A1
(43) Pub. Date: May 3, 2007

(54) Cosmetic application of rod-coil copolymers

(76) Inventors: Nathalie Mougin, Paris (FR); Xavier Schultze, Pontault-Combault (FR)

Correspondence Address: OLIFF & BERRIDGE, PLC, P.O. BOX 19928, ALEXANDRIA, VA, 22320, US

(21) Appl. No.: 10/582,668

(22) PCT Filed: Dec. 10, 2004
(86) PCT No.: PCT/FR04/50679

§ 371 Date: Nov. 17, 2006

Related U.S. Application Data
(60) Provisional application No. 60/534,999, filed on Jan. 9, 2004.

(30) Foreign Application Priority Data
Dec. 11, 2003 (FR) 03 51028

(51) Int. Cl. A61K 8/64

(52) U.S. Cl. 424/70.14; 424/78.3

(57) ABSTRACT

The present invention relates to a cosmetic composition containing, in a physiologically acceptable medium, at least one rod-coil type block copolymer comprising at least one “coil” polymeric block structure of variable conformation bonded to at least one “rod” block structure of restricted conformation, the composition being characterized in that said rod block structure is of polymeric nature and is constituted in full or in part of peptide motifs or the like with some or all of the free hydrogen atoms of said peptide motifs participating in non-covalent hydrogen bonds within the rod structure.

The present invention relates more particularly to proposing the application of rod-coil type block copolymers in the field of cosmetics.

In general, polymers are compounds frequently used in cosmetic compositions in order to give them properties that are improved either in terms of formulation, and/or durability, and/or comfort for users.

Thus, numerous compositions and in particular compositions for hair styling which are presented in the form of very fine droplets, gels, or foams, contain resins or polymers.

These are in particular acrylic polymers having vitreous glass transition temperatures (Tg) that are high. Such polymers serve in particular to enable hair styles to hold better. Nevertheless, their excessive friability does not enable this holding to be guaranteed over a long duration. An alternative then consists in associating them with compounds known as “plasticizers” that serve to lower the glass transition temperature. Unfortunately, polymers then tend to manifest a sticky effect and/or to become less good-at styling, i.e. they tend to lose some of their mechanical properties.

Polymers also constitute ingredients that are important in the field of nail varnishes. They contribute in particular to forming the film of varnish that is applied to the surface of the nails. Nevertheless, as in the above application, the protective film, which contains the polymer, does not always turn out to present sufficient resistance to the mechanical abrasion to which varnished nails are subjected daily.

Consequently, there is at present no satisfactory solution for conferring properties on a cosmetic composition by means of polymers that give satisfaction simultaneously in terms of mechanical strength and in terms of not being sticky.

Unexpectedly, the inventors have found that rod-coil type block copolymers of specific composition do indeed make it possible for to all of these requirements to be satisfied.

The polymers considered in the present invention are easily applied to the hair, and surprisingly, they present satisfactory styling power and can easily be removed, merely with shampoo. Brushing the treated hair does not lead to a powder effect, but leaves the hair soft and shiny and not sticky.

Rod-coil block copolymers are copolymers associating firstly one or more sequences or blocks of polymers forming a stick-shape, referred to as a “rod block”, and characterized by a tendency to assemble automatically in an anisotropic structure, and secondly one or more sequences or blocks of polymers in the form of random tangles referred to as “coil blocks”, that can take on multiple shapes, unlike a rod block. These two types of block, rod and coil, are bonded together covalently.

Rod-coil block copolymers are characterized by very high miscibility due to the large incompatibility of the respective rod and coil blocks, and they are specifically advantageous in this respect for obtaining microstructures and nanostructures with large separate phases.

International patent application WO 99/47570 thus proposes using rod-coil block copolymers in a solvent that is selective for only one of the two blocks, so as to dissolve the block concerned and thus enable rod-coil type copolymers to self-assemble in organized mesostructures each with a region of non-dissolved blocks and a region of dissolved blocks.

Mesostructures of that type are described in particular as being advantageous for encapsulating active materials within the mesostructure, or for forming absorption layers at the surface of a substrate.

The rod blocks that are proposed more particularly in patent application WO 99/47570 are polymers of a chemical structure that incorporates a plurality of aromatic rings such as quinoline, pyridine, thiophene, and benzymidazole, for example.

It is the Pi interactions, also known as “Pi-Pi stacking”, generated between these aromatic rings that enable the rod blocks to be rigid. More precisely, the origin of the weak interaction that holds each cycle parallel is the overlapping Pi bonds between two aromatic groups with electron displacement. This interaction produces an energy minimum that stabilizes the structure and leads to the rod blocks being oriented in parallel.

Unexpectedly, the inventors have found that it is possible to reproduce these advantageous properties manifested by rod polymer blocks as described above while making use of another type of interaction that is not covalent.

More precisely, in one of its aspects, the present invention-provides a cosmetic composition containing, in a physiologically acceptable medium, at least one rod-coil type block copolymer comprising at least one “coil” polymeric block structure of variable conformation bonded to at least one “rod” block structure-of restricted conformation, the composition being characterized in that said rod block structure is of polymeric nature and is constituted in full or in part of peptide motifs or the like with some or all of the free hydrogen atoms of said peptide motifs participating in non-covalent hydrogen bonds within the rod structure.

In another of its aspects, the present invention also relates to the use of rod-coil block copolymers in accordance with the invention as surface active agents and/or as Theological agents.

These copolymers can also be used for their mechanical properties, as film-forming polymers when mixed with other polymers, as matrix reinforcement, or indeed, when mixed with molecules, as supports for encapsulating them, e.g. molecules such as coloring agents and/or anti-UV agents.

In another of its aspects, the invention provides a rod-coil type block copolymer comprising at least one polymer block structure of variable conformation referred to as “coil” bonded to at least one block structure of limited conformation referred to as “rod”, the block copolymer being characterized in that said rod block structure is of polymeric nature and made up in part or in full of peptide or analogous units, with all or some of the free hydrogen atoms of said peptide units participating in non-covalent hydrogen bonds within the rod structure, and in that said coil block is made up of:

radical homo- or copolymers derived from radical polymerization of at least one ethylene monomer of the butadiene, or (meth)acrylic, (meth)acrylamide, allyl, vinyl alcohol ester, or vinyl ether type;

polycondensates of polyurethane and/or polyureas, aliphatic polyesters, aliphatic polyamides, or copolymers thereof, such as, for example, poly(urethane/urea) and poly(ester/amide)polycondensates;

polymers obtained by opening cycles selected from polyesters such as polycaprolactone; and polyoxazolines such as poly(2-methyloxazoline), or poly(2-ethyloxazoline);

homopolymers of siloxane, such as, for example polydimethylsiloxane (PDMS), polymethylphenylsiloxane, and polymethyllaurylsiloxane;

polymers obtained by metathesis such as poly(norbornene) and copolymers thereof;

copolymers comprising monomers obtained by cationic polymerization such as polyvinylalkylethers, e.g. polyvinylmethylethers;

copolymers of different types of the above-specified polymers, such as, for example poly(urethane siloxane);

copolymers of different types of the above-mentioned polymers together with other copolymers such as, for example, polysiloxane and ethylene polyoxyde copolymers; and

salts and derivatives thereof.

Rod-Coil Block Copolymer

The copolymers implemented in compositions in accordance with the invention turn out to be particularly effective in creating nanoscopic organization in a solvent or other medium. The rod-coil molecular architecture implies both a phase separation of rod blocks and coil blocks within structures at submicron scale, due to repulsion from each of the types of block, and volume constraints, due to the covalent bonding imposed by the connectivity of each of the blocks and also by the organization of the rod blocks.

They thus turn out to be capable of automatically assembling in bulk in a variety of supramolecular structures such as lamellar, hexagonal, micellar, and vesicular structures, for example.

Similarly, when they are put in the presence of a solvent, they can assemble in a variety of supramolecular structures by interactions between the solvent and the polymer segments. In this implementation, structures of nanoscopic scale are obtained that advantageously possess improved mechanical properties for low viscosity, in particular viscosity less than 1 pascal-second (Pa·s) or 1,000 centipoises (cps).

Viscosity is measured at a concentration of 20% by weight of polymer in water, and at 25° C., using a spindle module Brookfield viscosimeter.

Viscosity may lie in particular in the range 10⁻³ Pa·s to 10 Pa·s (1 cps to 10,000 cps) and in particular in the range 10⁻³ Pa·s to 5 Pa·s (1 cps to 5,000 cps), or indeed 5×10⁻³ Pa·s to 3 Pa·s (5 cps to 3,000 cps).

The formation of a specific morphology can be controlled, in obvious manner given the nature of the blocks, but also the overall mass of the polymer, the relative length of the blocks, the nature of the solvent, the concentration of the polymer, additives, and temperature.

The respective chemical natures of the rod and coil blocks, and in particular their incompatibility encourage phase separation. The rigidity of one of the two blocks can also encourage such phase separation.

Conventionally, the morphology adopted by such systems depends on the Flory-Huggins interaction coefficient χ between the various blocks, and thus on their chemical natures, on the degree of polymerization, and on the volume fraction occupied by each block. In general, the longer the chains, the more phase separation is encouraged. A difference in rigidity between the rod chains and the coil chains thus leads to an increase in the interaction coefficient χ and thus encourages phase separation and nanostructuring in the solid state.

Rod-coil block copolymers of the invention comprise at least one self-organized rod block and at least one coil block.

In the meaning of the present invention, the term “block” covers a repetitive chain of monomer units with this repetition being equal to at least two units, and in particular to three units, and in particular being not less than five units or better not less than seven units.

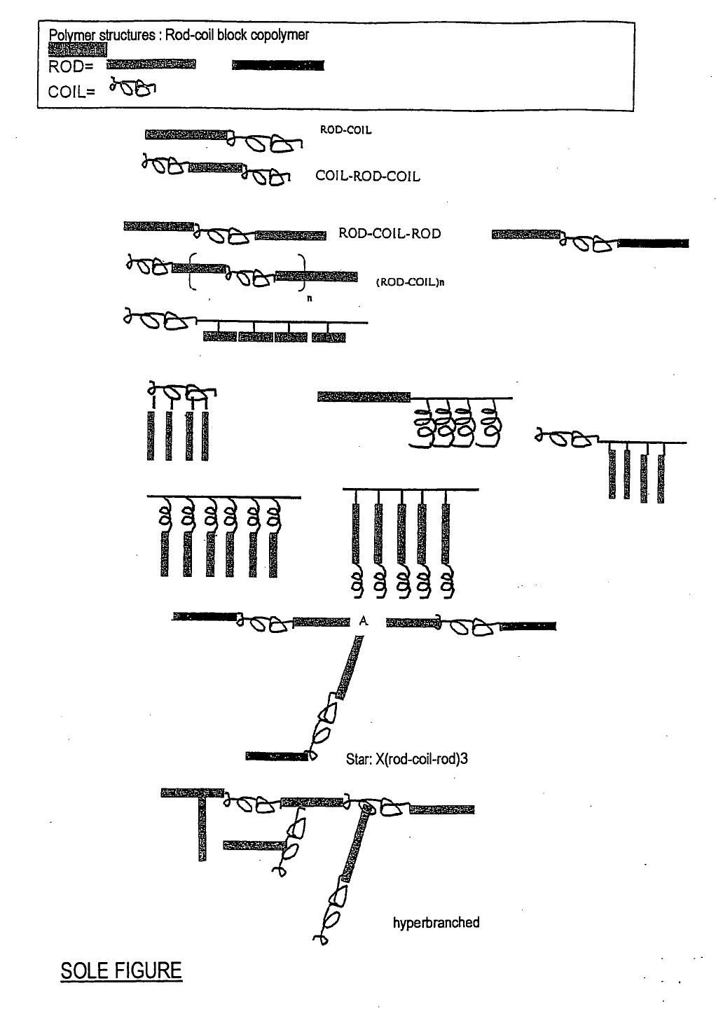

The rod-coil block copolymers of the invention may present a variety of architectures, in particular AB type di-blocks where A relates to the rod block and B to the coil block, ABA or BAB or ABC type tri-blocks, or (AB)ₙ or (ABC)ₙ multiblocks, where C designates a rod or coil block of a kind different from A and B. The polymer may also be branching, having a skeleton made up of the rod sequence or the polymer may be branching with rod type grafts, or the polymer may be in a star configuration. By way of illustration that is not limiting on the invention, FIG. 1 shows several possible types of structure for copolymers in accordance with the invention.

The overall number average molecular mass of the rod-coil copolymers of the invention generally lies in the range 700 grams per mole (g/mol) to 1,000,000 g/mol, in particular 1,000 g/mol to 800,000 g/mol, and more particularly 2,000 g/mol to 500,000 g/mol.

Copolymers of the invention may include at least one cross-linkable sequence. In a variant of the invention, they are advantageously implemented in non-cross-linked form.

Rod-coil block copolymers turn out to be particularly advantageous because of their useful mechanical properties at low mass and thus because of their low viscosity compared with “conventional” polymers that need to have high molecular weights in order to obtain the necessary mechanical properties.

Furthermore, rod-coil block copolymers turn out to be entirely compatible with being applied in the form of a spray. They are thus particularly advantageous in this respect for use in the field of hair care.

Finally, when they possess an amphiphilic nature, they can easily be conveyed in a hydrosoluble or liposoluble medium because of the sequence that gives them the desired solubility.

Rod Block

Rod blocks of the invention present trajectory persistence, i.e. orientational anisotropy which implies a limited degree of freedom for the chains.

The pseudo-rigid or even rigid conformation of the rod blocks is provided mainly by non-covalent interactions imposing a direction on the chain and, in the invention, due to the existence of non-covalent hydrogen bonds manifested by the hydrogen atoms of the peptide motifs present in the rod blocks.

In the meaning of the present invention, a peptide motif is constituted by at least a first amino acid molecule bonded by a peptide bond, also known as an amine bond, to a second amino acid molecule that can be identical or different. In other words, these motifs are characterized by the presence in their structure of at least one peptide bond having formula (I):

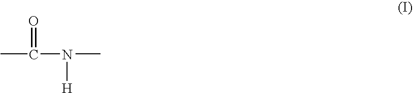

The interactions of the non-covalent hydrogen bond type generated by these peptide bonds, when sufficient in number and/or strategically placed, serve to limit in significant manner the number of possible ways in which the various monomers making up the rod block can be arranged in three dimensions relative to one another. Thus, the mean length of the chain taking account of the various possible conformations is greater: all the restrictions imposed on the free hinging of the monomers relative to one another have the effect, on average, of stretching the chain and thus of increasing the mean distance between the ends of the chain represented by <R₀²>_{rod} and capable of satisfying the following convention:
<R₀²>_{rod}═CN L²
where:

L represents the length of a monomer;

C represents the restrictions imposed on the chain with C greater than 1, and in particular lying in the range 4 to 10; and

N represents the number of monomers constituting the block.

By associating these rod blocks with coil blocks, a large amount of conformational asymmetry is introduced into a given macromolecule. It is this asymmetrical conformation that encourages incompatibility and thus improves the conductive forces for nanoscopic separation and thus self-assembly of the rod-coil block copolymers.

In a preferred variant of the invention, the rod block polymer structure satisfies in full or in part the following general formula (II) or (III): 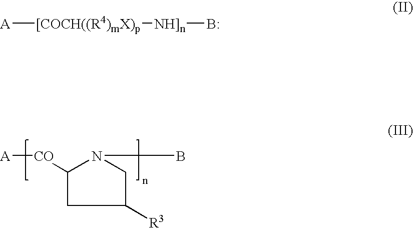
in which:

n represents an integer number not less than 3, in particular lying in the range 5 to 1,000, and more particularly 7 to 500;

m represents an integer in the range 0 to 30;

p represents an integer in the range 0 to 1;

X represents:
OR¹, and in particular OH;
N R¹ R², and in particular NH₂;
COOR¹, and in particular COOH;
NH(C) (NH)NR¹R²;
a heterocycle, optionally condensed, possibly including 1 to 2 nitrogen atoms and being unsaturated, such as for example an indole or a pyrrole radical;
S R¹;
OCOR¹;
OCONR¹R²;
OCOOR¹;
CONR¹R²;
NR¹COR²;
NR¹COO R²;
PO(OR¹)₂;
SS R¹;
SCOR¹;
SCOO R¹; or
SO₃H;

R¹, R², and R³ representing independently of one another:
a hydrogen atom;
a linear alykl grouping, in particular in the range C₁ to C₁₂ grouping, more particularly C₂ to C₈, branched, where appropriate cyclic; or
an aromatic grouping, in particular aryl, aralkyl, alkylaryl, or diaryl, in the range C₁ to C₃₀, e.g. of the benzyl type;

R⁴ represents a divalent linear alkylene grouping in particular in the range C₁ to C₁₂, more particularly C₂ to C₈, that is branched, possibly cyclic, or an aromatic grouping, in particular arylene, aralkylene, or alkylarylene, or diarylene in the range C₁ to C₃₀, e.g. of the benzylene type

with R⁴, m, and X possibly having respective different meanings within the general formula (II);

A represents a hydroxyl or derived function, represents the bond established with a coil block structure, or represents a function capable of initiating peptide polymerization, such as, for example R¹ NH—;

B represents a hydrogen atom or represents the bond established with a coil block structure; and

derivatives thereof.

The general formulae (II) and (III) thus define copolypeptides constituted by monomers that are generally not identical, that are distributed in random or block manner, or that alternate in the chain.

In the meaning of the present invention, said alkyl, aryl, aralkyl, alkylaryl, or diaryl groupings include, where appropriate, an O, N, P, or S heteroatom, e.g. in the form of an alcohol, ether, amine, amide, ester, or acid function, and the hydrogen atoms may be substituted in full or in part by fluorine atoms.

In the meaning of the present invention, the term “derivatives” covers salts and their substitution derivatives, such as for example, methyl, ether, amide, and ester derivatives.

The term “salts” is used to mean salified species of amines or acids. The salt formation may be performed using organic or inorganic salts.

With acids, the neutralizing agent may be an inorganic base, such as LiOH, NaOH, KOH, Ca(OH)₂, NH₄OH, or an organic base such as, for example: a primary, secondary, or tertiary alkylamine such as butylamine or triethylamine, said primary, secondary, or tertiary alkylamine possibly including atoms of nitrogen and/or oxygen, and thus including for example an alcohol function, for example such as amino-2-methyl-2-propanol or triethanolamine.

With amines, suitable neutralizing agents are the salts of mineral acids such as sulfuric acid or hydrochloric acid, or the salts of organic acids. These organic acids may include one or more carboxylic, sulfonic, or phosphonic acid groups. There may be thus be linear or branched or cyclic aliphatic acids, or indeed aromatic acids. The acids may also include one or more heteroatoms selected from O and N, e.g. in the form of hydroxyl groups.

An example of an alkyl group acid is acetic acid CH₃COOH.

An example of a polyacid is terephthalic acid.

Examples of hydroxyacids include citric acid and tartaric acid.

Where appropriate, the interactions of the invention of the non-covalent hydrogen bond type can be replaced by interactions of other types relating to steric repulsion, Lewis acid/base type coordination, acid/base interactions, dipole/dipole interactions, for example.

By way of illustration of systems presenting steric repulsion, particular mention can be made of poly(ethylsiloxane) and poly(triphenylacrylate).

The rod blocks of the invention can thus be constituted solely by peptide motifs as defined above, or they may include in their structure motifs of a different chemical nature capable of initiating a non-covalent interaction of some type other than hydrogen bonds.

Most particularly appropriate are polymers of formula (II) in which:

m represents an integer in the range 0 to 12;

X represents a grouping selected from —O R¹, —NR¹R², —COOR¹, —NR¹COR², —CONR¹R², —NR¹COO R², or —SR¹; with R¹ and R² representing independently of each other a hydrogen atom, a methyl, ethyl, propyl, butyl, isobutyl, isopropyl, phenyl, benzyl, trifluoromethyl, —(CH₂)₂OH or —(CH₂)₃OH group.

More particularly, the rod block structure is derived from homopolymerization or copolymerization of one or more amino acids selected from the group consisting in glycine, alanine, phenylalanine, valine, isoleucine, leucine, arginine, asparagine, aspartic acid, cysteine, methionine, glutamine, glutamic acid, histidine, lysine, serine, threonine, tryptophane, tyrosine, proline, and derivatives thereof.

The compound of general formula (III) is representative in particular of the homogeneous motif of the proline or hydroxyproline type and monosubstitution derivatives thereof.

By way of non-limiting illustration of rod block polymers in accordance with the invention, mention can be made in particular of:

poly(L-leucine), poly(L-valine), poly(phenylalanine);

poly(L-lysine) and derivatives thereof, such as poly(N-benzyloxycarbonyl-L-lysine) and poly(N-trifluoroacetyl-L-lysine), and derivatives thereof such as hydrochlorides;

poly(L-glutamic acid) and salts thereof such as the sodium salt, and derivatives thereof, such as poly γ-alkylesters in the range C₁-C₃₀ of L-glutamic acid such as poly(γ-methyl-L-glutamate) or poly γ-aryl esters in the range C₁-C₃₀ or poly γ-alkyl aryl esters in the range C₁-C₃₀ such as poly(γ-benzyl-L-glutamate);

polyglutamine and derivatives thereof, such as poly(N-hydroxyethyl-L-glutamine and poly (N-hydroxypropyl-L-glutamine); and

polypeptide copolymers of the above monomers of the poly(hydroxyethyl-L-glutamine and leucine), poly (hydroxyethyl-L-glutamine and valine), poly (γ-benzyl-L-glutamate and leucine), poly(γ-benzyl-L-glutamate and D, L-phenylalanine, poly (γ-benzyl-L-glutamate and cinnamylglutamate), poly(N-benzyloxycarbonyl-L-lysine and γ-benzyl-L-glutamate) type, and salts and derivatives thereof.

It is possible to control the hydrophilic or hydrodispersable nature of a rod block by selecting the monomers making it up.

Thus, rod blocks of the poly(glutamic acid) and poly(L-lyzine) type and salts thereof are more particularly hydrophilic.

In a particular variant of the invention, the number average molecular mass of the rod blocks lies in the range 200 g/mol to 1,000,000 g/mol, in particular 250 g/mol to 800,000 g/mol, and more particularly 250 g/mol to 500,000 g/mol.

In a particular variant of the invention, the rod blocks are present at at least 10%, in particular at least 15%, or at least 30%, and in particular at most 90%, or at most 85%, or at most 80% by weight relative to the total weight of the copolymer.

This ratio may be conditioned in particular by the desired solubility. Thus, if the coil block is soluble in the desired medium while the rod block is not, then the proportion of the first type of coil block can advantageously be adjusted to a greater value.

Coil Block

Compared with the above-described rod blocks, a coil block is constituted by a homopolymer or a copolymer in which the chain presents a geometrical conformation that is randomly variable. The degrees of freedom of the chemical bonds between the monomers making up the chain are thus considerably more numerous for a coil block than for a rod block, thereby leading to a considerably larger number of possible conformations.

Compared with the rod block, the mean distance between the ends of a chain in the coil block, i.e. <R₀²>_{coil} satisfies the convention:
<R₀² >_{coil}═N L²
where N and L are as defined above.

The coil blocks of the rod-coil copolymers used in compositions of the invention are generally made up of radical homopolymers or copolymers derived from radical polymerization of ethylene monomers, in particular vinyl monomers; and in particular including at least one (meth)acrylic, (meth)acrylamide, allylic, vinyl alcohol ester, and/or vinyl ether grouping.

Amongst these monomers, particular mention can be made of those that satisfy the following formulae.

(i) (meth)acrylates having the formula: 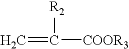
in which:

R₂ is hydrogen or methyl (CH₃); and

R₃ represents:

a linear, cyclic, or branched alkyl group having 1 to 30 carbon atoms, in which there may be found one or more heteroatoms selected from O, N, S, and P; said alkyl group possibly also being substituted by one or more substituents selected from —OH, halogen atoms (Cl, Br, I, and F), and the groups —Si(R₄R₅R₆) and —Si(R₄R₅)O, in which R₄, R₅, and R₆ are identical or different and represent a hydrogen atom, an alkyl group in the range C₁ to C₆, or a phenyl group;

in particular R₃ may be a methyl, ethyl, propyl, n-butyl, isobutyl, tertiobutyl, hexyl, ethylhexyl and in particular ethyl-2-hexyl, octyl, lauryl, isooctyl, isodecyl, dodecyl, cyclohexyl, t-butylcyclohexyl or stearyl, ethyl-2-perfluorohexyl, ethyl-2-perfluorooctyl group, or a hydroxyalkyl group in the range C₁-C₄ such as 2-hydroxyethyl, 2-hydroxybutyl and 2-hydroxypropyl; or a (C₁₋₄)alcoxy or a (C₁₋₄)alkyl such as methoxyethyl, ethoxyethyl and methoxypropyl;

a cycloalkyl group in the range C₃ to C₁₂, such as the isobornyl group;

a C₃ to C₂₀ aryl group such as the phenyl group;

a C₄-C₃₀ aralkyl group (C₁-C₈ alkyl group) such as 2-phenyl-ethyl, t-butylbenzyl, or benzyl;

a heterocyclic group having 4 to 12 links containing one or more heteroatoms selected from O, N, and S, the cycle being aromatic or otherwise; and

a heterocycloalkyl group (C₁ to C₄ alkyl), such as furfurylmethyl or tetrahydrofurfurylmethyl; said cycloalkyl, aryl, aralkyl, heterocyclic, or heterocycloalkyl groups optionally being substituted by one or more constituents selected from hydroxyl groups, halogen atoms, and C₁ to C₄ alkyl groups that may be linear or branched, in which there may optionally be included one or more heteroatoms selected from O, N, S, and P, said alkyl groups possibly also being substituted by one or more substituents selected from —OH, halogen atoms (Cl, Br, I and F), and the groups —Si(R₄R₅R₆) and —Si(R₄R₅)O groups, in which R₄, R₅ and R₆ are identical or different and represent a hydrogen atom, a C₁ to C₆ alkyl group, or a phenyl group;

a —(OC₂H₄)ₘ—OR″ group with m=5 to 300 and R″═H or a C₁ to C₃₀ alkyl group, e.g. —(OC₂H₄)ₘ—OH, —(OC₂H₄)ₘ-methyl, or —(OC₂H₄)ₘ-behenyl; a —(OC₃H₆)ₘ—OR″ group with m=5 to 300 and R″═H, or a C₁ to C₃₀ alkyl group, e.g. —(OC₃H₆)ₘ—OH; or indeed a random or block mixture of the groups (OC₂H₄)ₘ and (OC₃H₆)ₘ.

(ii) (meth)acrylamides having the formula: 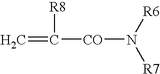
in which:

R₈ designates H or methyl; and

R₇ and R₆ are identical or different and represent;
a hydrogen atom; or
a linear or branched alkyl group having 1 to 30 carbon atoms, in which there may be found one or more heteroatoms selected from O, N, S, and P; said alkyl group possibly also being substituted by one or more substituents selected from —OH, halogen atoms (Cl, Br, I, and F), and the groups —Si(R₄R₅R₆) and —Si(R₄R₅)O, in which R₄, R₅, and R₆ and represent a hydrogen atom, an alkyl group in the range C₁ to C₆, or a phenyl group;
in particular R₃ may be a methyl, ethyl, propyl, n-butyl, isobutyl, tertiobutyl, hexyl, ethylhexyl, octyl, lauryl, isooctyl, isodecyl, dodecyl, cyclohexyl, t-butylcyclohexyl or stearyl, ethyl-2-perfluorohexyl, ethyl-2-perfluorooctyl group, or a hydroxyalkyl group in the range C₁-C₄ such as 2-hydroxyethyl, 2-hydroxybutyl and 2-hydroxypropyl; or a (C₁₋₄)alcoxy or a (C₁₋₄)alkyl such as methoxyethyl, ethoxyethyl and methoxypropyl;
a cycloalkyl group in the range C₃ to C₁₂, such as the isobornyl group;
a C₃ to C₂₀ aryl group such as the phenyl group;
a C₄-C₃₀ aralkyl group (C₁-C₈ alkyl group) such as 2-phenyl-ethyl, t-butylbenzyl or benzyl;
a heterocyclic group having 4 to 12 links containing one or more heteroatoms selected from O, N, and S, the cycle being aromatic or otherwise; and
a heterocycloalkyl group (C₁ to C₄ alkyl), such as furfurylmethyl or tetrahydrofurfurylmethyl; said cycloalkyl, aryl, aralkyl, heterocyclic or heterocycloalkyl groups optionally being substituted by one or more constituents selected from hydroxyl groups, halogen atoms, and C₁ to C₄ alkyl groups that may be linear or branched, in which there may optionally be included one or more heteroatoms selected from O, N, S, and P, said alkyl groups possibly also being substituted by one or more substituents selected from —OH, halogen atoms (Cl, Br, I and F), and the groups —Si(R₄R₅R₆) and —Si(R₄R₅)O groups, in which R₄, R₅ and R₆ are identical or different and represent a hydrogen atom, a C₁ to C6 alkyl group, or a phenyl group;
a —(OC₂H₄)ₘ—OR″ group with m=5 to 300 and R″═H or a C₁ to C₃₀ alkyl group, e.g. —(OC₂H₄)ₘ—OH, —(OC₂H₄)ₘ-methyl, or —(OC₂H₄)ₘ-behenyl; a —(OC₃H₆)ₘ—OR″ group with m=5 to 300 and R″═H, or a C₁ to C₃₀ alkyl group, e.g. —(OC₃H₆)ₘ—OH; or indeed a statistical or block mixture of the groups (OC₂H₄)ₘ and (OC₃H₆)ₘ.

Examples of (meth)acrylamide monomers are (meth)acrylamide, N-ethyl(meth)acrylamide, N-butylacrylamide, N-t-butylacrylamide, N-isopropylacrylamide, N,N-dimethyl(meth)acrylamide, N,N-dibutylacrylamide, N-octylacrylamide, N-dodecylacrylamide, N-undecylacrylamide, and N-(2-hydroxypropylmethacrylamide).

(iii) Vinyl compounds having the formula:
CH₂═CH—R₉
in which:

R₉ is a hydroxyl group; halogen (Cl or F); an NH₂ group; an acetamide group (NHCOCH₃); or R₉ is selected from:

a linear or branched alkyl group having 1 to 30 carbon atoms, in which there may be found one or more heteroatoms selected from O, N, S, and P; said alkyl group possibly also being substituted by one or more substituents selected from —OH, halogen atoms (Cl, Br, I, and F), and the groups —Si(R₄R₅R₆) and —Si(R₄R₅)O, in which R₄, R₅, and R₆ are identical or different and represent a hydrogen atom, an alkyl group in the range C₁ to C₆, or a phenyl group;

a cycloalkyl group in the range C₃ to C₁₂, such as the isobornyl group;

a C₃ to C₂₀ aryl group such as the phenyl group;

a C₄-C₃₀ aralkyl group (C₁-C₈ alkyl group) such as 2-phenylethyl, or benzyl;

a heterocyclic group having 4 to 12 links containing one or more heteroatoms selected from O, N, and S, the cycle being aromatic or otherwise, such as N-vinylpyrrolidone and N-vinylcaprolactam; and

a heterocycloalkyl group (C₁ to C₄ alkyl), such as furfurylmethyl or tetrahydrofurfurylmethyl; said cycloalkyl, aryl, aralkyl, heterocyclic, or heterocycloalkyl groups optionally being substituted by one or more constituents selected from hydroxyl groups, halogen atoms, and C₁ to C₄ alkyl groups that may be linear or branched, in which there may optionally be included one or more heteroatoms selected from O, N, S, and P, said alkyl groups possibly also being substituted by one or more substituents selected from —OH, halogen atoms (Cl, Br, I and F), and the groups —Si(R₄R₅R₆) and —Si(R₄R₅)O groups, in which R₄, R₅ and R₆ are identical or different and represent a hydrogen atom, a C₁ to C₆ alkyl group, or a phenyl group.

Examples of vinyl monomers are vinylcyclohexane, and styrene, N-vinylpyrrolidone, and N-vinylcaprolactam.

(iv) Vinyl ethers having the formula R₆O—CH═CH₂ or vinyl esters having the formula:
R₆—C(O)O—CH═CH₂
in which:

R₆ represents a linear or branched alkyl group having 1 to 22 atoms, a cyclic alkyl group having 3 to 12 carbon atoms, a phenyl group, a C₃-C₂₀ aryl group, or C₄-C₃₀ arallyl group.

Examples of vinyl esters are vinyl acetate, vinyl propionate, vinyl butyrate, vinyl ethylhexanoate, vinyl neononanoate, and vinyl neododecanoate.

Among vinyl ethers, mention can be made of vinylmethylether, vinylethylether, and vinylisobutylether.

(v) Allyl compounds having the formula:
CH₂═CH—CH₂—R₉ or CH₂═C(CH₃)—CH₂—R₉
in which R₉ has the same meaning as above.

Mention can be made in particular of allylmethylether, 3-allyloxy-1,2-propanediol (CH₂═CHCH₂OCH₂CH(OH)CH₂OH), and 2-allyloxyethanol (CH₂═CHCH₂OC₂H₄OH).

(vi) Ethylenically unsaturated monomers comprising at least one carboxylic, phosphoric, or sulfonic acid function or anhydride, such as for example acrylic acid, methacrylic acid, crotonic acid, maleic anhydride acid, itaconic acid, fumaric acid, maleic acid, styrenesulfonic acid, vinylbenzoic acid, vinylphosphoric acid, acrylamidopropanesulfonic acid, and salts thereof.

Amongst the salts, mention can be made of those obtained by neutralizing acid groups using inorganic bases such as LiOH, NaOH, KOH, Ca(OH)₂, NH₄OH, or Zn(OH)₂; or by an organic base such as primary, secondary, or tertiary alkylamine, in particular triethylamine or butylamine. The primary, secondary, or tertiary alkylamine may include one or more atoms of nitrogen and/or oxygen and may thus include one or more alcohol functions, for example; mention can be made in particular of amino-2-methyl-2-propanol, triethanolamine, and dimethylamino-2-propanol. Mention can also be made of lysine or of 3-(dimethylamino)-propylamine.

Mention can also be made of the salts of mineral acids such as sulfuric acid, hydrochloric acid, hydrobromic acid, hydroiodic acid, phosphoric acid, and boric acid. Mention can also be made of the salts of organic acids, which may include one or more carboxylic, sulfonic, or phosphonic acid groups. It is also possible to use linear, branched, or cyclic aliphatic acids, or indeed aromatic acids. These acids may further include one or more heteroatoms selected from O and N, e.g. in the form of hydroxyl groups. Mention can be made in particular of propionic acid, acetic acid, terephthalic acid, citric acid, and tartaric acid.

The following copolymers are suitable for the invention: vinyl and (meth)acrylate, vinyl and (meth)acrylamide, vinyl and (meth)acrylate and (meth)acrylamide, olefin and vinyl, and (meth)acrylate and (meth)acrylamide copolymers, and also (meth)acrylate and (meth)acrylate copolymers.

To illustrate these polymers, mention can be made more particularly of homopolymers of ethylene polyoxide(meth)acrylate, of stearyl(meth)acrylate, of lauryl(meth)acrylate, of butyl(meth)acrylate, of ethylhexyl(meth)acrylate, of crotonic acid, of (meth)acrylic acid, of maleic anhydride, of sulfonic styrene acid, of dimethyldiallylamine, of dimethylaminoethyl(meth)acrylate, of dimethylaminopropyl(meth)acrylamide, and salts thereof.

Mention can also be made of copolymers based on vinyl acetate, styrene, vinylpyrrolidone, vinylcaprolactam, ethylene polyoxide(meth)acrylate, stearyl(meth)acrylate, lauryl(meth)acrylate, vinyl laurate, butyl(meth)acrylate, ethylhexyl(meth)acrylate, crotonic acid, (meth)acrylic acid, maleic anhydride, sulfonic styrene acid, dimethyldiallylamine, vinylpyridine, dimethylaminoethyl(meth)acrylate, dimethylaminopropyl(meth)acrylamide, and salts thereof.

More particularly, mention can be made of copolymers of acrylic acid and butyl acrylate; copolymers of dimethylaminoethyl acrylate and methyl(meth)acrylate; copolymers of dimethylaminoethyl methacrylate and methyl(meth)acrylate; copolymers of (meth)acrylic acid and butyl acrylate; copolymers of methacrylic acid and ethylene polyoxide methacrylate; copolymers of ethylene polyoxide methacrylate and methyl acrylate.

The coil blocks of the polymers used in the compositions of the invention may also be made of:

polycondensates of polyurethane and/or polyureas, of aliphatic polyesters, of aliphatic polyamides or of their copolymers, such as for example polycondensates of poly(urethane/urea) and poly(ester/amide);

polymers obtained by cycle opening, selected from polyethers of the ethylene polyoxide type, propylene polyoxide and copolymers thereof, polylactides, polyesters such as polycaprolactone; and polyoxazolines such as poly(2-methyloxazoline), or poly(2-ethyloxazoline);

homopolymers of siloxane, such as polydimethylsiloxane (PDMS), polymethylphenylsiloxane, and polymethyllaurylsiloxane;

polymers obtained by metathesis such as poly(norbornene) and copolymers thereof;

polymers obtained by cationic polymerization such as polyvinylalkylethers e.g. polyvinylmethylethers;

copolymers of different types of the above polymers;

copolymers of different types of the above polymers with other polymers, such as for example polysiloxanes and ethylene polyoxide copolymers; and

salts and derivatives thereof.

Where appropriate, these polymers may be functionalized so as to confer a soluble or dispersible nature thereon, in particular in the solvent in which are they are to be formulated, such as for example, water and/or ethanol, or carbon, ester, fluorinate, or silicone oils.

In the meaning of the invention, a polymer is said to be soluble if it forms a liquid solution, and is said to be dispersible if at 5% by weight in the solvent in question and at 25° C., it forms a stable suspension of fine particles that are generally spherical.

Particularly suitable to the invention in terms of. coil blocks, are blocks that are soluble or dispersible in water and/or ethanol, and that may include anionic, cationic, and/or hydrophilic non-ionic motifs. Polymers including anionic or cationic groups can be implemented in optionally neutralized form.

Anionic groups can be neutralized using an inorganic base such as LiOH, NaOH, KOH, Ca(OH)₂, NH₄OH or an organic base as defined above.

Cationic groups can be neutralized with an acid such as sulfuric acid or hydrochloric acid, or an organic acid as defined above.

The cationic groups can be quarternized by mobile halogen compounds such as chlorides or bromides of C₁-C₁₂ alkyls, and in particular methyl bromide or ethyl chloride, by sodium chloroacetate, or by cyclic sulfones, e.g. propanesulfone.

Representative of polymers having non-ionic motifs, mention can be made in particular of polymers based on ethylene oxide, on methyloxazoline, on ethyloxazoline, on vinylpyrrolidone, on vinylcaprolactam, and on ethylene polyoxide(meth)acrylate.

As anionic polymers, mention can be made more particular of homopolymers or copolymers of ethylene based on crotonic acid, (meth)acrylic acid, maleic anhydride, styrene sulfonic acid, aliphatic sulfonic polyesters, aliphatic sulfonic polyamides, and salts thereof.

As cationic polymers, mention can be made more particularly of ethylene homopolymers or copolymers based on dimethyldiallylamine, on vinylpyridine, on dimethylaminoethyl(meth)acrylate, on dimethylaminopropyl(meth)acrylamide, on tertiary aliphatic amine polyesters, on tertiary aliphatic amine polyamides, and salts thereof.

Also suitable for the invention are liposoluble coil copolymers.

As representative of non-hydrosoluble and liposoluble polymers forming a coil block, mention can be made of:

polydimethylsiloxanes (PDMS) and copolymers based on phenyl(polymethylphenylsiloxane), and/or on fluorides that are soluble in silicones or derivatives thereof;

optionally hydrogenated polybutadienes, polyisobutylenes, and (ethylene/butylene)copolymers soluble in mineral oils;

alkyde polyesters having C₆ to C₃₀ carbon chains;

polyamides having C₆ to C₃₀ carbon chains;

vinyl polymers based on vinyl laurate;

poly(meth)acrylates based on stearyl(meth)acrylate, isobornyl(meth)acrylate, cyclohexane(meth)acrylate, butyl(meth)acrylate, ethylhexyl(meth)acrylate, and/or behenyl(meth)acrylate.

In copolymers of the invention, the number average molecular mass-of the coil block(s) may lie in the range 300 g/mol to 1,000,000 g/mol, in particular 500 g/mol to 800,000 g/mol, and more particularly 500 g/mol to 500,000 g/mol.

Synthesizing Rod Coil Block Copolymers

Rod-coil block polymers of the invention can be obtained by various synthesis techniques.

It is possible to use a reaction between two free-formed rod and coil precursors involving the condensation reaction between a functional end of a rod block and a functional end of a coil block. This may be constituted, in particular, by a reaction of esterification, of (trans)esterification, of (trans)amidification, forming urethane bonds, ureas, C—C, C═C, or C≡C bonds, or imine bonds.

Concerning the preparation of rod blocks in accordance with the invention, the synthesis technique that is most used for obtaining homo-polypeptides, and also co-polypeptides, consists in opening the corresponding N-carboxyanhydride α-amino acid (NCA) in the presence of a nucleophile such as a primary amine or a strong base such as sodium alkoxyde. This technique is described in particular in B. Gallot, Prog. Polym. Sci., 1035, 21, 1996 and by T. J. Deming, Macromol. 2970, 35, 2002. Certain transition metals can also be used for achieving controlled polymerization of NCA as described in patent application WO 01/94379.

Concerning the synthesis of coil blocks, reference can be made in particular to the document “Chimie et physicochimie des polymers” [Chemistry and physicochemistry of polymers], published by Dunod 2002.

In another variant, one of the two blocks constituting the rod-coil block copolymer may be made in situ in the presence of the other block that has already been constituted. For example, if the coil block is a polymer that can be obtained by the radical technique, it suffices to functionalize one of the ends of the rod block with a function capable of initiating controlled radical polymerization of the monomers under consideration for the coil block. Similarly, the rod block may be formed in situ in the presence of a coil block and on a reactive function of that block.

Thus, when the coil block is a copolymer derived from polymerizing monomers having ethylene motifs, it can be obtained by anionic polymerization and functionalization can be performed by trapping the active anion with a molecule of the following type: 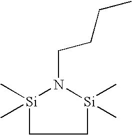
so as to generate a protected amine function at the end of the chain that is subsequently deprotected so as to become capable of initiating polymerization of N carboxyanhydride.

The same applies if the copolymer is derived from radical polymerization. The amine function is then attached to the chain via the initiator of so-called controlled radical polymerization. By way of example, it may be H₂N-phenyl-OC(O)C(CH₃)(CH₃)Br. The Br function is capable of initiating polymerization of radical monomers by the ATRP method.

Depending on the functions of the precursors and the distribution of the functions, it is possible to obtain di-blocks, tri-blocks, multiblocks, branched polymers or stars.

As an illustration of rod-coil block copolymers of the invention, mention can be made initially of:

rod-block-coil (also abbreviated as rod-b-coil) di-block, such as for example ethylene polyoxide-b-poly(gamma benzyl L-glutamate), poly(N-benzyloxycarbonyl-L-lysine)-b-ethylene polyoxide, ethylene polyoxide-b-poly(gamma L-glutamic acid), polyL-lactide-b-poly(gamma benzyl L-glutamate), ethylene polyoxide-b-poly(gamma benzyl L-glutamate), poly(gamma benzyl lysine)-b-polystyrene, polydimethylsiloxane-b-poly(L glutamic acid), acrylic polyacid-b-poly(L-glutamic acid), sulfonic polyester-b-poly(L-glutamic acid), poly(gamma methyl-L glutamate)-b-polyurethane, polyethylene imine-b-poly(L-phenylalanine), polymethyloxazoline-b-poly(L-phenylalanine), polycaprolactone-b-poly(L-alanine), polycaprolactone-b-polyglycine, polybutadiene-b-poly(L-glutamic acid), polydimethylsiloxane-b-poly(benzyl-glutamate), poly(N-benzyloxycarbonyl-L-lysine)-b-poly(ethylene oxide co propylene oxide), poly(ethylene oxide co propylene oxide)-b-poly gamma L-glutamic acid, poly(ethylene oxide co propylene oxide)-b-poly(gamma benzyl L-glutamate), and salts thereof;

coil-block-rod-block-coil or rod-b-coil-b-rod tri-blocks such as for example poly(gamma benzyl L-glutamate)-b-polystyrene-b-poly(gamma benzyl L-glutamate), poly(L-glutamic acid)-b-polybutadiene -b-poly(L-glutamic acid), poly(L-glutamic acid)-b-polydimethylsiloxane-b-poly(L-glutamic acid), poly(gamma benzyl L-glutamate)-b-poly(ethylene oxide or co propylene oxide)-b-poly(gamma benzyl L-glutamate), poly(benzyl-glutamate)-b-polydimethylsiloxane-b-poly(benzyl-glutamate), poly(L-glutamic acid)-b-acrylic polyacid-b-poly(L-glutamic acid), poly(L-glutamic acid)-b-sulfonic polyester-b-poly(L-glutamic acid), poly(gamma methyl-L glutamate)-b-polyurethane-b-poly(gamma methyl-L glutamate; poly(L-phenylalanine)-b-imine polyethylene-b-poly(L-phenylalanine), poly(L-phenylalanine)-b-polymethyloxazoline-b-poly(L-phenylalanine), poly(L-alanine)-b-polycaprolactone-b-poly(L-alanine), polyglycine-b-polycaprolactone-b-polyglycine, poly(L-valine)-b-poly(ethylene oxide co propylene oxide)-b-poly(L-valine), and salts thereof.

As specified above, certain rod-coil copolymers suitable for the invention constitute as such an aspect of the invention.

More particularly, these copolymers are as defined in claim 35.

Their rod segment(s) satisfy the above definition while their coil segment(s) are made up of:

radical homo- or copolymers derived from radical polymerization of at least one ethylene monomer of at least one of the following types: butadiene, (meth)acrylic, (meth)acrylamide, allyl, vinyl alcohol ester, and vinyl ether;

polycondensates of polyurethane and/or polyureas, of aliphatic polyesters, of aliphatic polyamides, or copolymers thereof, such as, for example, polycondensates of poly(urethane/urea) and poly(ester/amide);

polymers obtained by opening cycles selected from polyesters such as polycaprolactone, and polyoxazolines such as poly(2-methyloxazoline), or poly(2-ethyloxazoline);

homopolymers of siloxane, such as for example polydimethylsiloxane (PDMS), and polymethylphenylsiloxane or polymethyllaurylsiloxane;

polymers obtained by metathesis such as poly(norbornene) and copolymers thereof;

copolymers comprising monomers obtained by cationic polymerization such as polyvinylalkylethers such as for example polyvinylmethylethers;

copolymers of different types of the above polymers such as, for example poly(urethane siloxane);

copolymers of different types of the above polymers with other copolymers such as, for example copolymers of polysiloxane and ethylene polyoxide; and

salts and derivatives thereof.

The rod segments are more particularly as defined above.

As representative and non-limiting rod-coil block polymers of the invention, mention can be made more particularly of those selected from:

rod-block-coil di-blocks such as poly(N-benzyloxycarbonyl L-lysine)-b-ethylene polyoxide, ethylene polyoxide-b-poly gamma L-glutamic acid, poly L-lactide-b-poly(gamma benzyl L-glutamate), ethylene polyoxide-b-poly(gamma benzyl L-glutamate), poly(gamma benzyl lysine)-b-polystyrene, polydimethylsiloxane-b-poly(L-glutamic acid), acrylic polyacid-b-poly(L-glutamic acid), sulfonic polyester-b-poly(L-glutamic acid), poly(gamma methyl L-glutamate)-b-polyurethane polyethylene imine-b-poly(L-phenylalanine), polymethyloxazoline-b-poly(L-phenylalanine), polycaprolactone-b-poly(L-alanine), polycaprolactone-b-polyglycine, polybutadiene-b-poly(L-glutamic acid)polydimethylsiloxane-b-poly(benzyl-glutamate), poly(N-benzyloxycarbonyl L-lysine)-b-poly(ethylene oxide co propylene oxide), poly(ethylene oxide co propylene oxide)-b-poly gamma L-glutamic acid, poly(ethylene oxide co propylene oxide)-b-poly(gamma benzyl L-glutamate), and salts thereof;

coil-block-rod-block-coil tri-blocks or rod-b-coil-b-rod tri-blocks such as poly(gamma benzyl L-glutamate)-b-propylene polyoxide-b-poly(gamma benzyl L-glutamate), poly(gamma benzyl L-glutamate)-b-polystyrene-b-poly(gamma benzyl L-glutamate), poly(L-glutamic acid)-b-polybutadiene-b-poly(L-glutamic acid), poly(L-glutamic acid)-b-polydimethylsiloxane-b-poly(L-glutamic acid), poly(L-glutamic acid)-b-polydimethylsiloxane-b-poly(L-glutamic acid)poly(gamma benzyl L-glutamate)-b-poly(ethylene oxide co propylene oxide)-b-poly(gamma benzyl L-glutamate), poly(benzyl-glutamate)-b-polydimethylsiloxane-b-poly(benzyl-glutamate), poly(L-glutamic acid)-b-acrylic polyacid-b-poly(L-glutamic acid), poly(L-glutamic acid)-b-sulfonic polyester-b-poly(L-glutamic acid), poly(gamma methyl-L glutamate)-b-polyurethane-b-poly(gamma methyl-L glutamate, poly(L-phenylalanine)-b-polyethylene imine-b-poly(L-phenylalanine), poly(L-phenylalanine)-b-polymethyloxazoline-b-poly(L-phenylalanine), poly(L-alanine)-b-polycaprolactone-b-poly(L-alanine), polyglycine-b-polycaprdlactone-b-polyglycine, poly(L-valine)-b-poly(ethylene oxide co propylene oxide)-b-poly(L-valine), and salts thereof.

Application of Rod-Coil Block Polymers

As mentioned above, rod-coil block polymers of the invention are particularly advantageous for their ability to lead to three-dimensional nanostructure systems in solution or in bulk. The interesting mechanical properties they display for small viscosity are also particularly advantageous.

They can be used for their ability to adsorb at interfaces, and in particular for their qualities as surface active agents and/or as a vehicle for encapsulating active agents.

Such systems may also present advantageous rheological qualities, and in particular they can increase the viscosity of the formulation that contains them.

They can also be used with advantage for their film-forming properties, or as additives for reinforcing organic or inorganic matrices.

In cosmetic compositions of the invention, rod-coil block copolymers are used in concentrations that are compatible with self-assembly in the form of a nanostructure. Such concentrations are generally concentrations greater than the critical micellar concentration or the critical vesicular concentration. These critical micellar or vesicular concentrations characterize concentrations beneath which the copolymer exists as an individual molecule or chain in the solution, and above which it exists as an aggregated species. These critical concentration values lie in the range 10⁻⁹ moles per liter (mol/L) to 10⁻⁴ mol/L.

Most particularly suitable are copolymer concentrations lying in the range 0.5% by weight to 90% by weight, in particular 0.7% by weight to 85% by weight, and more particularly 0.8% by weight to 75% by weight of copolymer relative to the total weight of the composition.

Physiologically Acceptable Medium

The term “physiologically acceptable medium” is used to mean a medium that is non-toxic and that is suitable for being applied to the skin, the lips, hair, or the nails of human beings. The physiologically acceptable medium is generally adapted to the surface on which the composition is to be applied and also to the way in which the composition is to be packaged, specifically whether it should be fluid or non-fluid at ambient temperature and under atmospheric pressure.

Aqueous Phase

The composition of the invention may include at least one aqueous medium, constituting an aqueous phase, capable of forming the continuous phase of the composition.

The aqueous phase may be constituted essentially by water.

It may also comprise a mixture of water and an organic solvent miscible in water (miscible in water to more than 50% by weight at 25° C.) such as lower monoalcohols having 2 to 5 carbon atoms such as ethanol, isopropanol, tertio-butanol, and n-butanol, and alkyleneglycols having 2 to 8 carbon atoms such as propylene glycol, 1,3-butylene glycol, dipropylene glycol, and C₃-C₄ ketones.

The aqueous phase (water and possibly the water-miscible organic solvent) may be present at a concentration lying in the range 1% to 95% by weight, in particular lying in the range 3% to 80% by weight, more particularly 5% to 6% by weight relative to the total weight of the composition.

Such a medium may also include at least one volatile oil as defined below.

Organic Solvents

The composition of the invention may comprise at least one organic solvent medium constituting an organic phase, made of at least one organic solvent that is volatile at ambient temperature. Such organic solvents are taken into consideration more particularly when the cosmetic composition is for application to the nails.

As an organic solvent that is optionally volatile at ambient temperature, mention can be made of the following:

ketones that are liquid at ambient temperature such as methylethylketone, methylisobutylketone, diisobutylketone, isophorone, cyclohexanone, and acetone;

alcohols that are liquid at ambient temperature such as ethanol, isopropanol, butanol, diacetone alcohol, 2-butoxyethanol, and cyclohexanol;

glycols that are liquid at ambient temperature such as ethylene glycol, propylene glycol, pentylene glycol, and glycerol;

propylene glycol ethers that are liquid at ambient temperature such as propylene glycol monomethylether, propylene glycol monomethyl ether acetate, and dipropylene glycol mono n-butyl ether;

short chain esters (having 3 to 8 carbon atoms in all) such as ethyl acetate, methyl acetate, propyl acetate, butyl acetate, aryl acetate, and isopentyl acetate;

alkanes that are liquid at ambient temperature such as decane, heptane, octane, dodecane, cyclohexane, and isododecane; and

mixtures thereof.

The composition of the invention may also contain one or more silicone oils, generally in small quantity, i.e. possibly less than 10% by weight of the solvent phase. In particular, it is possible to use optionally volatile oils such as dimethicone, phenyldimethicone, alkyldimethicone, dimethicone, dimethicone copolyol, or cyclomethicone.

The above organic compounds can be present at a concentration of 0.5% to 99% by weight relative to the total weight of the composition.

When the physiologically acceptable medium includes a significant quantity of organic phase, it may represent 30% to 99% by weight and in particular 60% to 90% by weight relative to the total weight of the composition.

Fatty Phase

The composition, in particular when it is for application to the lips or the skin may include at least one fatty phase and in particular at least one fat that is liquid at ambient temperature (25° C.) and at atmospheric pressure and/or at least one fat that is solid at ambient temperature and at atmospheric pressure such as waxes, pasty fats, gums, and mixtures thereof. The fatty phase may also contain agents for gelling and structuring oils of organic nature and/or lipophilic organic solvents.

The composition may possess, for example, a continuous fatty phase, possibly containing less than 5% water, in particular less than 1% water relative to the total weight of the composition and in particular it may be in anhydrous form.

As a liquid fat, the fatty phase of the composition of the invention may comprise in particular at least one volatile or non-volatile oil, or a mixture thereof.

The term “volatile oil” is used in the invention to mean any oil liable to evaporate on being in contact with the skin for less than 1 hour, at ambient temperature, and at atmospheric pressure. Volatile oils of the invention are cosmetic volatile oils, that are liquid at ambient temperature, having non-zero vapor pressure at ambient temperature and atmospheric pressure, in particular lying in the range 1.01 millimeters of mercury (mmHg) to 300 mmHg (i.e. 1.33 pascals (Pa) to 40,000 Pa), and preferably being greater than 0.3 mmHg (30 Pa).

The term “non-volatile oil” is used to mean an oil that remains on the skin at ambient temperature and at atmospheric pressure for at least several hours and in particular having a vapor pressure of less than 0.01 mmHg (1.33 Pa).

These volatile or non-volatile oils may be hydrocarbon oils in particular of vegetable origin, silicone oils, or mixtures thereof. The term “hydrocarbon oil” is used to mean an oil containing mainly atoms of hydrogen and of carbon, possibly together with atoms of oxygen, nitrogen, sulfur, and/or phosphorus.

The volatile hydrocarbon coils can be selected from hydrocarbon oils having 8 to 16 carbon atoms, in particular C₈-C₁₆ branched alkanes such as C₈-C₁₆ isoalkanes of petroleum origin (also known as isoparaffins), such as isododecane (also known as 2,2,4,4,6-pentamethylheptane), isodecane, isohexadecane, and for example the oils sold under the trade names Isopars® or Permetyls®, C₈-C₁₆ branched esters such as isohexyl neopentanoate, and mixtures thereof. Other volatile hydrocarbon oils such as petroleum distillates, in particular those sold under the name Shell Solt® by the supplier Shell, can also be used.

As volatile oils, it is also possible to use volatile silicones, such as for example volatile linear or cyclic silicone oils, in particular those having viscosity≦8 centistokes (8×10⁻⁶ m²/s), and having in particular 2 to 7 silicon atoms, these silicones optionally including alkyl or alkoxy groups having 1 to 10 carbon atoms. As a volatile silicone oil suitable for use in the invention, mention can be made in particular of octamethyl cyclotetrasiloxane, decamethyl cyclotetrasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane, and mixtures thereof.

The volatile oil may be present in the composition of the invention at a concentration lying in the range 0.1% to 98% by weight, in particular 1% to 65% by weight, and more particularly 2% to 50% by weight relative to the total weight of the composition.

The non-volatile oils may be selected in particular from hydrocarbon oils that are fluorinated, where appropriate, and/or non-volatile silicone oils.

As non-volatile hydrocarbon oils, mention can be made in particular of the following:

hydrocarbon oils of animal origin;

hydrocarbon oils of vegetable origin such as triglycerides constituted by fatty acid esters and glycerol in which the fatty acids can have a variety of chain lengths in the range C₈ to C₂₄, said chains possibly being linear or branched, saturated or unsaturated; these oils are in particular the following oils: wheat germ, sunflower, grape seed, sesame, maize, apricot, castor, karite, avocado, olive, soy bean, sweet almond, palm, colza, cotton, hazelnut, macadamia, jojoba, alfalfa, poppy, Hokkaito squash, sesame, vegetable marrow, colza, black current, evening primrose, millet, barley, quinoa, rye, safflower, candlenut, passion fruit, muscat rose tree; karite butter; or indeed triglycerides of caprylic and capric acids such as those sold by the supplier Stearineries Dubois or those sold under the names Miglyol 810®, 812®, and 818® by the supplier Dynamic Nobel:

synthetic ethers having 10 to 40 carbon atoms;

linear or branched hydrocarbons of mineral or synthetic origin such as Vaseline, polydecanes, hydrogen polyisobutene such as parleam, squalene, and mixtures thereof;

synthetic esters such as oils having the formula R¹COOR² in which R¹ represents the residue of a linear or branches fatty acid having 1 to 40 carbon atoms and R² represents a hydrocarbon chain, in particular a branch chain containing 1 to 40 carbon atoms on the condition that R¹+R² is greater than or equal to 10, for example Purcellin oil (cetostearyl octanoate), isopropyl myristate, isopropyl palmitate, benzoates of C₁₂ to C₁₅ alcohols, hexyl laurate, diisopropyl adipate, isononyl isononanoate, 2-ethyl-hexyl palmitate, isostearyl isostearate, heptanoates, octanoates, decanoates, or ricinoleates of alcohols or of polyalcohols such as propylene glycol dioctanoate; hydroxyl esters such as isostearyl lactate, di-isostearyl malate; polyol esters, and pentaerythritol esters;

fatty alcohols that are liquid at ambient temperature having a branched and/or unsaturated carbon chain with 12 to 26 carbon atoms such as octyl dodecanol, isotearylic alcohol, oleic alcohol, 2-hexyldecanol, 2-butyloctanol, and 2-undecylpentadecanol; and

higher fatty acids such as oleic acid, linoleic acid, linolenic acid, and mixtures thereof.

The non-volatile silicone oils usable in the composition of the invention may be non-volatile polydimethylsiloxanes (PDSM), polydimethylsiloxanes including alkyl or alcoxy groupings that are pendant and/or at the ends of the silicone chain, groupings each having 2 to 24 carbon atoms, phenyl silicones such as phenyl trimethicones, phenyl dimethicones, phenyl trimethylsiloxy diphenylsiloxanes, diphenyl dimethicones, diphenyl methyldiphenyl trisiloxanes, and 2-phenylethyl trimethylsiloxysilicates.

The non-volatile oils may be present in the composition of the invention at a concentration lying in the range 0.01% to 90% by weight, in particular 0.1% to 85% by weight, and more particularly 1% to 70% by weight, relative to the total weight of the composition.

More generally, the fat that is liquid at ambient temperature at atmospheric pressure may be present at a concentration of 0.01% to 90% by weight, and in particular 0.1% to 85% by weight relative to the weight of the fatty phase.

Concerning the fat that is solid at ambient temperature and at atmospheric pressure, it can be selected from waxes, pasty fats, gums, and mixtures thereof. This solid fat may be present at a concentration of 0.01% to 50%, particularly 0.1% to 40%, and more particularly 0.2% to 30% by weight relative to the total weight of the composition.

Thus, the composition of the invention may include at least one fatty compound that is pasty at ambient temperature.

In the meaning of the invention, the term “pasty fat” covers fats having a melting point lying in the range 20° C. to 55° C., in particular 25° C. to 45° C., and/or viscosity at 40° C. lying in the range 0.1 Pa·s to 40 Pa·s (1 poise to 400 poises), in particular 0.5 Pa·s to 25 Pa·s, measured using a Contraves TV or a Rheomat 80 instrument, with a moving body turning at 60 hertz (Hz). The person skilled in the art can select the moving body for measuring viscosity amongst those referenced MS-r3 and MS-r4, on the basis of general knowledge, in particular in order to be able to measure the viscosity of the pasty compound being tested.

More particularly, these fats may be hydrocarbon compounds, possibly of the polymeric type; they may also be selected from silicone compounds; they may also be in the form of a mixture of hydrocarbon and/or silicone compounds. When there is a mixture of different pasty fats, it is preferable to use hydrocarbon pasty compounds (mainly containing atoms of carbon and hydrogen, possibly together with ester groupings), as a majority proportion.

Amongst the fatty compounds suitable for use in the composition of the invention, mention can be made of lanolins and lanolin derivatives such as acetyl lanolins, oxypropylene lanolins, or isopropyl lanolate, having viscosity in the range 18 Pa·s to 21 Pa·s, preferably 19 Pa·s to 20.5 Pa·s, and/or a melting point of 30° C. to 55° C., and mixtures thereof. It is also possible to use esters of acid or of fatty alcohols, in particular those having 20 to 65 carbon atoms (melting point about 20° C. to 35° C. and/or viscosity at 40° C. lying in the range 0.1 Pa·s to 40 Pa·s) such as cetyl or tri-isotearyl citrate; arachidyl propionate; vinyl polylaurate; esters of cholesterol such as triglycerides of vegetable origin such as hydrogenated vegetable oils, viscous polyesters, and mixtures thereof. As a triglyceride of vegetable origin, it is possible to use derivatives of hydrogenated castor oil, such as Thixinr® from Rheox.

It is also possible to mention silicone pasty fats such as polydimethylsiloxanes (PDMS) of high molecular weight, and in particular those having pendant chains of the alkyl or alcoxy type with 8 to 24 carbon atoms, a melting point of 20° C.-55° C., such as stearyl dimethicones, in particular sold by the supplier Dow Corning under the trade name DC2503® and DC25514®, and mixtures thereof.

The pasty fats may be present in the composition of the invention at a concentration lying in the range 0.01% to 50% by weight, in particular lying in the range 0.1% to 45% by weight, and more particularly 0.2% to 30% by weight, relative to the total weight of the composition.

The composition of the invention may also include a wax. The wax may be solid at ambient temperature (25° C.), with reversible liquid/solid change of state, having a melting temperature greater than 30° C. and possibly as high as 200° C., hardness greater than 0.5 MPa, and in the solid state presenting crystal organization that is anisotropic. It may be a hydrocarbon wax, a fluorocarbon wax, and/or a silicone wax, and it may be of animal, vegetable, mineral, or synthetic origin. By way of example it may be selected from beeswax, carnauba wax, Candelilla wax, paraffin wax, hydrogenated castor oil wax, silicone waxes, microcrystalline waxes, and mixtures thereof.

In particular, the wax may be present in the form of a wax-in-water emulsion.

The wax may be present in the composition of the invention at a concentration lying in the range 0.01% to 50% by weight, in particular 0.01% to 30% by weight, and more particularly 0.2% to 20% by weight, relative to the total weight of the composition.

Surface Active Agents

The composition of the invention may also contain emulsion-forming surface active agents present in particular at a concentration lying in the range 0.1% to 30% by weight, better 5% to 15% by weight, relative to the total weight of the composition.

The surface active agents may be selected from surface active agents that are anionic, cationic, amphoteric, or non-ionic. Reference can be made to the document “KIRK-OTHMER Encyclopedia of Chemical Technology”, Vol. 22, pp. 333-432, 3rd edition, 1979, Wiley, for a definition of the properties and the (emulsion forming) functions of surface active agents, in particular on pages 347-377 of that reference, for anionic and non-ionic surface active agents.

The surface active agents that are preferably used in the composition of the invention are selected from:

non-ionic surface active agents such as fatty acids and fatty alcohols; and/or

anionic surface active agents.

Particulate Phase

The composition of the invention may also include an additional particulate phase present at a concentration of 0.01% to 40% by weight, in particular 0.01% to 30% by weight and more particularly 0.05% to 20% by weight relative to the total weight of the composition.

In particular, it may include at least one complementary pigment, nacre, and/or filler of the kind used in cosmetic compositions.

Pigments should be understood as comprising particles that are white or colored, mineral or organic, insoluble in the liquid hydrophilic phase, and intended to give the composition color and/or covering power. Fillers should be understood as particles that are colored or white, mineral or synthetic, lamellar or non-lamellar. Nacres should be understood as being iridescent particles, in particular those produced by certain mollusks in their shells, or else synthesized.

The pigments may be present in the composition at a concentration of 0.01% to 25% by weight, in particular 0.01% to 15% by weight, and more particularly 0.02% to 5% by weight relative to the weight of the composition.

Mineral pigments suitable for use in the invention include the oxides of titanium, zirconium, or cerium, and also the oxides of zinc, of iron, or chromium, ferric blue, manganese violet, ultramarine blue, and chromium hydrate. Organic pigments that are suitable for use in the invention include carbon black, C&D type pigments, and lakes based on cocheneal carmine, barium, strontium, calcium, aluminum, or indeed the diketopyrrolopyrroles (DPPs) described in patent documents EP-A-0 542 669, EP-A-0 787 730, EP-A-0 787 731, and WO-A-96/08537. The quantity and/or the selection of these pigments are generally varied to take account of the quantity of nanotubes present in the cosmetic composition under consideration.

Nacres can be present in the composition at a concentration of 0.01% to 25% by weight, in particular 0.01% to 15% by weight, and more particularly 0.02% to 5% by weight, relative to the total weight of the composition.

Nacreous pigments can be selected from white nacreous pigments such as titanium-covered mica, or bismuth oxychloride, colored nacreous pigments such as titanium mica with iron oxides, titanium mica in particular with ferric blue or with chromium oxide, titanium mica with an organic pigment of the type mentioned above, and nacreous pigments based on bismuth oxychloride.

The additional fillers may be present at a concentration of 0.01% to 40% by weight, in particular 0.01% to 30% by weight, and more particularly 0.02% to 20% by weight relative to the total weight of the composition. Their quantity is also generally adjusted to take account of the quantity of nanotubes.

The filler may also be constituted by a spherical filler such as, for example: talc, zinc stearate, mica, kaolin, powders of polyamide (Nylon®) (orgasol® from Atochem), polyethylene powders, powders of tetrafluoroethylene polymers (Teflon®), starch, boron nitride, polymeric microspheres such as those of polyvinylidene chloride and acrylonitrile such as Expancel® (Nobel Industrie), copolymers of acrylic acid (Polytrap® from the supplier Dow Corning), microbeads of silicone resin (Tospearls® from Toshiba, for example), and organopolysiloxanes elastomers.

The composition may also include hydrosoluble or liposoluble coloring agents at a concentration lying in. the range 0.01% to 6% by weight, relative to the total weight of the composition, and more particularly in the range 0.01% to 3% by weight. By way of example, the liposoluble coloring agents may be Sudan red, DC red 17, DC green 6, β-carotene, soy bean oil, Sudan brown, DC yellow 11, DC violet 2, DC orange 5, and quinoline yellow. Hydrosoluble coloring agents may be constituted, for example, by beetroot juice and by methylene blue.

In addition, the composition of the invention may include all of the conventional ingredients used in the fields concerned, and more particularly in the fields of cosmetics and dermatology. These ingredients are in particular selected from vitamins, antioxidants, thickening agents, oligo-elements, softeners, sequestering agents, fragrances, alkaline or acidic agents, preservatives, UV filters, hydrophilic or lipophilic active agents, and mixtures thereof. The quantities of these various ingredients are those that are conventional in the fields concerned, e.g. lying in the range 0.01% to 20% by weight of the total weight of the composition.

Naturally, the person skilled in the art will select any such additional ingredients and/or the quantities thereof in such a manner that the advantageous properties of the composition of the invention are not, or at least not significantly, spoiled by the addition in question.

The composition of the invention may be obtained using the methods of preparation that are conventional in cosmetics or dermatology.

The composition may be in the form of an aqueous or an oily solution, an oil-in-water emulsion, or a water-in-oil emulsion.

It may also be in the form of a product that has been cast into a stick or a cake such as a lipstick or balm, a cake of foundation, antiwrinkle compositions, complexion “correctors” and/or “beautifiers”, makeup for the eyelids or for the cheeks.

It may be in the form of a care product and/or makeup for the nails, the skin, and/or the lips.

It may also be in the form of a composition for application to the hair for care and/or styling purposes, whether in the form of a lacquer, spray, gel, foam, shampoo, or conditioner.

It may be formulated in the presence of a propellant which may be any liquefiable gas of the kind commonly used-in aerosol devices.

The present invention also provides a method of cosmetically treating at least one keratinous material, in particular the skin, the hair, and/or the nails, comprising applying a composition of the invention on said material.

The examples of compositions given below are given by way of non-limiting illustration.

### EXAMPLE 1

1) Preparing the propylene polyoxide-block-poly(gamma benzyl-L-glutamate) block polymer;

2.454 grams (g) of (gamma)benzyl-L-glutamate-N-carboxyanhydride were dissolved in 60 g of dichloromethane, under argon; and

0.875 g of alpha, omega NH₂polypropylene glycol (4000 g/mol) dissolved in 4 g of dichloromethane were added to said solution using a syringe.

The reaction took place over 5 days at ambient temperature.

The reaction medium was then precipitated in ethyl ether.

A white powder was obtained which was dried in a vacuum giving 1.86 g of polymer, i.e. a yield of about 62%.

2) Preparing the polypropylene glycol-block-poly(L-glutamic acid) polymer by hydrolyzing the benzyl grouping:

1.86 g of the above synthesized polymer were placed in a reflux in 15 millimeters (mL) of trifluoroacetic acid for 4 hours (h).

Thereafter the polymer was precipitated in ethyl ether and dried under a vacuum.

1.2 g of polymer were recovered, giving a hydrolysis yield of 70% measured by nuclear magnetic resonance (NMR)

### EXAMPLE 2

Polyaminoacid-b-poly(ethylene oxide co propylene oxide)-b-polyaminoacid.

-α, ω̅ diNH₂-poly(ethylene oxide co propylene oxide)-(diNH₂-P(OE/PO)) having a mass of 1000 g.mol⁻¹ (1 g) was put into solution in 10 mL of chloroform.

The reaction medium was swept under nitrogen. 0.86 g of valine-NCA were then added over 2 h: gas (CO₂) was observed to be given off. The reaction was left at ambient temperature for 24 h.

That method gave rise to the following polymers:

| | | | | Composition of resulting |
|---|---|---|---|---|
| | | | Mass ratio^{d}: | polymer: coil/rod^{e} |
| | Pre-polymer | NCA^{c} | diNH₂P(OE/PO)/NCA | mass ratio |
| Example 2.1 | diNH₂ P(OE/PO)^{a}: 2 g | Valine: 0.86 g | 70/30 | 84/16 |
| Example 2.2 | diNH₂ P(OE/PO)^{a}: 1.514 g | Benzyl-glutamate: 1.816 | 45/55 | 55/54 |

^{a}−α,ω diNH₂-poly(ethylene oxide co propylene oxide) with a molar ratio of PO/EO = 3/19; a mass of 1000 g · mol⁻¹.
^{c}Derived from N-carboxyanhydride: (see drawing below).
^{d}Mass ratio in the starting mixture.
^{e}Mass ratio in the polymer after purification, determined by NMR.

### EXAMPLE 3

Using the same method as described above the following polymers were obtained:

| | | | | Composition of resulting |
|---|---|---|---|---|
| | | | Mass ratio^{d}: | polymer: coil/rod^{e} |
| | Pre-polymer | NCA^{c} | diNH₂PDMA/NCA | mass ratio |
| Example 3.1 | diNH₂PDMS^{b}: 0.28 g | Benzyl-glutamate: 1.35 g | 17/83 | 84/16 |

^{b}−α,ω diNH₂ -polydimethylsiloxane of mass 10,000 1000 g · mol⁻¹.
^{c}N-carboxyanhydride of structures given below.
^{d}Mass ratio of the starting mixture.
^{e}Mass ratio in the polymer after purification, as determined by NMR.

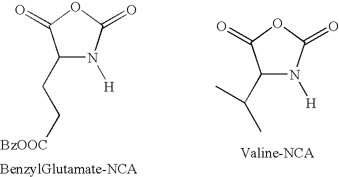

### EXAMPLE 4

Cosmetic Composition for Hair

The compound of Example 1 was dissolved in water at 10% by weight by being dissolved directly.

The solution was put into a pump flask and projected onto the hair. The spray produced a non-sticky hair styling composition.

### EXAMPLE 5

Cosmetic Composition for the Hair

The compound of Example 2.2 was dissolved in water at 10% by weight by being dissolved directly.

The solution was put into a pump flask and projected onto the hair. The spray gave a non-sticky hair styling composition.

### EXAMPLE 6

Cosmetic Composition for the Nails

The compound of Example 3.1 was dissolved to 20% by weight in ethyl acetate.

The solution was applied to the nails.

A shiny film was thus obtained that was not sticky.

1. A cosmetic composition, comprising at least one rod-coil type block copolymer comprising at least one “coil” polymeric block structure of variable conformation bonded to at least one “rod” block structure of restricted conformation, wherein:
the at least one rod-coil type block copolymer is provided in a physiologically acceptable medium, and
the rod block structure is of polymeric nature and is constituted in full or in part of peptide motifs with some or all of the free hydrogen atoms of the peptide motifs participating in non-covalent hydrogen bonds within the rod structure.

2. The composition according to claim 1, wherein the non-covalent hydrogen bonds within the rod structure are present in sufficient number and/or are strategically placed as to ensure that the rod polymeric structure has a mean distance between the ends of its chain <R₀²> satisfying the convention:
<R₀²>_{rod}═CNL²
where:
L represents a length of a monomer;
C represents restrictions imposed on the chain and is greater than 1; and
N represents a number of monomers constituting the block.

3. A composition according to claim 1, wherein the rod polymeric block structure satisfies general formula (II) or (III), or is a derivative thereof: 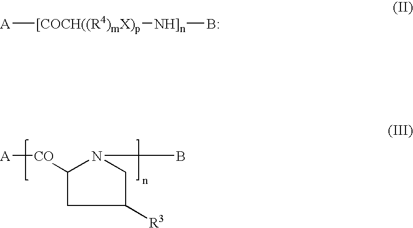
in which:
n represents an integer not less than 3;
m represents an integer from 0 to 30;
p represents an integer from 0 to 1:
X represents:
OR¹;
NR¹R²;
COOR¹;
NH—C(NH)NR¹R²;
an optionally condensed heterocycle optionally including 1 to 2 nitrogen atoms and being unsaturated;
SR¹;
OCOR¹;
OCONR¹R²;
OCOOR¹;
CONR¹R²;
NR¹COR²;
NR¹COO R²;
PO(OR¹)₂;
SSR¹;
SCOR¹;
SCOOR¹; or
SO₃H;
R¹, R², and R³ represent independently of one another:
a hydrogen atom;
a linear branched or cyclic alkyl group; or
an aromatic group;
R⁴ represents a divalent linear, branched or cyclic-alkylene group or an aromatic group;
A represents a hydroxyl or derived function, represents the bond established with a coil block structure, or represents a function capable of initiating peptide polymerization; and
B represents a hydrogen atom or represents the bond established with a coil block structure;

4. The composition according to claim 3, wherein the rod-coil block polymer satisfies formula (II) in which:
m represents an integer from 0 to 12;
X represents a group selected from —OR¹, —NR¹R², —COOR¹, —NR¹COR², —CONR¹R², —NR¹COOR², or —SR¹; and
R¹ and R² represent independently of each other a hydrogen atom, a methyl, ethyl, propyl, butyl, isobutyl, isopropyl, phenyl, benzyl, trifluoromethyl, —(CH₂)₂OH or —(CH₂)₃OH group.

5. The composition according to claim 1, wherein the rod block structure is derived from homopolymerization or copolymerization of one or more amino acids selected from the group consisting of glycine, alanine, phenylalanine, valine, isoleucine, leucine, arginine, asparagine, aspartic acid, cysteine, methionine, glutamine, glutamic acid, histidine, lysine, serine, threonine, tryptophane, tyrosine, proline, and derivatives thereof.

6. The composition according to claim 1, wherein the rod-block structure is selected from the group consisting of:
poly(L-leucine), poly(L-valine), poly(phenylalanine);
poly(L-lysine)
poly(L-glutamic acid) and salts thereof;
polyglutamine
polypeptide copolymers selected from the group consisting of poly(hydroxyethyl-L-glutamine and leucine), poly(hydroxyethyl-L-glutamine and valine), poly(γ-benzyl-L-glutamate and leucine), poly(γ-benzyl-L-glutamate and D,L-phenylalanine, poly(γ-benzyl-L-glutamate and cinnamylglutamate), poly(N-benzyloxycarbonyl-L-lysine and γ-benzyl-L-glutamate), and salts; and
derivatives thereof.

7. The composition according to claim 1, wherein a number average molecular mass of the rod blocks is from 200 g/mol to 1,000,000 g/mol.

8. The composition according to claim 1, wherein the rod blocks are present in an amount of at least 10%, by weight relative to a total weight of the copolymer.

9. The composition according to claim 1, wherein a mean distance between the ends of a chain in the coil block, satisfies the convention:
<R₀²>_{coil}═NL²
where:
L represents a length of a monomer; and
N represents a number of monomers constituting the block.

10. The composition according to claim 1, wherein the coil block is made of one or more copolymers or homopolymers derived from radical polymerization of monomers comprising ethylene, vinyl, allyl, (meth)acrylate, and/or (meth)acrylamide motifs and derivatives thereof.

11. The composition according to claim 1, wherein the coil polymer is selected from the group consisting of:
vinyl and (meth)acrylate copolymers, vinyl and (meth)acrylamide copolymers, vinyl and (meth)acrylate and (meth)acrylamide copolymers, olefin and vinyl copolymers, (meth)acrylate and (meth)acrylamide copolymers, and (meth)acrylate and (meth)acrylate copolymers.
homopolymers or copolymers based on at least one of vinyl acetate, styrene, vinylpyrrolidone, vinylcaprolactam, ethylene polyoxide(meth)acrylate, stearyl(meth)acrylate, lauryl(meth)acrylate, vinyl laurate, butyl(meth)acrylate, ethylhexyl(meth)acrylate, crotonic acid, (meth)acrylic acid, maleic anhydride, sulfonic styrene acid, dimethyldiallylamine, vinylpyridine, dimethylaminoethyl(meth)acrylate, and dimethylaminopropyl(meth)acrylamide;
polycondensates of at least one of polyurethane, polyureas, aliphatic polyesters, aliphatic polyamides;
polymers obtained by cycle opening, selected from the group consisting of polyethers of the ethylene polyoxide type, propylene polyoxide and copolymers thereof, polylactides, polyesters, and polyoxazolines;
homopolymers of siloxane;
polymers obtained by metathesis;
polymers obtained by cationic polymerization; and
copolymers, salts and derivatives thereof.

12. The composition according to claim 1, wherein a number average molecular mass of the coil block is from 300 g/mol to 1,000,000 g/mol.

13. The composition according to claim 1, wherein an overall number average molecular mass of the rod-coil copolymer is from 700 g/mol to 1,000,000 g/mol.

14. The composition according to claim 1, wherein the rod-coil block copolymer is not cross-linked.

15. The composition according to claim 1, wherein the rod-coil copolymer is selected from the group consisting of:
rod-block-coil di-blocks,
coil-block-rod-block-coils, rod-b-coil-b-rod tri-blocks and salts thereof.

16. The composition according to claim 1, wherein composition contains the copolymer in an amount of from 0.5% to 90% by weight, relative to a total weight of the composition.

17. The composition according to claim 1, wherein the composition comprises at least one aqueous phase.

18. The composition according to claim 1, wherein the composition comprises at least one fatty phase.

19. The composition according to claim 1, wherein the composition is anhydrous.

20. The composition according to claim 18, wherein the fatty phase comprises at least one of a fat that is liquid at ambient temperature and at atmospheric pressure, and a fat that is solid at ambient temperature and at atmospheric pressure.

21. The composition according to claim 20, wherein the fat that is liquid at ambient temperature and at atmospheric pressure comprises at least one volatile or non-volatile oil or a mixture thereof.

22. The composition according to claim 20, wherein the fat that is liquid at ambient temperature and at atmospheric pressure is present in an amount of from 0.01% to 90% by weight, relative to a total weight of the fatty phase.

23. The composition according to claim 20, wherein the fat that is solid at ambient temperature and at atmospheric pressure is selected from waxes, pasty fats, gums, and mixtures thereof.

24. The composition according to claim 18, wherein the fatty phase comprises at least one solid fat in an amount of form 0.01% to 50%, by weight relative to a total weight of the composition.

25. The composition according to claim 1, wherein the composition further comprises a particulate phase in an amount of 0.01% to 40%, by weight relative to a total weight of the composition.

26. The composition according to claim 25, wherein the particulate phase comprises at least one of an additional pigment, nacre or filler.

27. The composition according to claim 1, wherein the composition is in the form of an oil-in-water or a water-in-oil emulsion.

28. The composition according to claim 1, wherein the composition is in the form of a product that has been cast as a stick or a cake.

29. The composition according to claim 1, wherein the composition is in the form of a makeup and/or a care product for the skin and/or the lips.

30. The composition according to claim 1, wherein the composition is in the form of a care product and/or a makeup for the nails.

31. The composition according to claim 1, wherein the composition is in the form of a care product and/or a styling composition for the hair.

32. A method of cosmetically treating a keratinous material comprising applying the composition according to claim 1 to the material.

33. A surface active agent, comprising at least one rod-coil type block copolymer comprising at least one “coil” polymeric block structure of variable conformation bonded to at least one “rod” block structure of restricted conformation, wherein the rod block structure is of polymeric nature and is constituted in full or in part of peptide motifs with some or all of the free hydrogen atoms of the peptide motifs participating in non-covalent hydrogen bonds within the rod structure.

34. A rheological agent, comprising at least one rod-coil type block copolymer comprising at least one “coil” polymeric block structure of variable conformation bonded to at least one “rod” block structure of restricted conformation, wherein the rod block structure is of polymeric nature and is constituted in full or in part of peptide motifs with some or all of the free hydrogen atoms of the peptide motifs participating in non-covalent hydrogen bonds within the rod structure.

35. A block copolymer of the rod-coil type comprising at least one “coil” polymeric block structure of variable conformation bonded to at least one “rod” block structure of restricted conformation, wherein:
rod block structure is of polymeric nature and is constituted in full or in part by peptide motif(s) with all or some of the free hydrogen atoms of the peptide motifs participating in non-covalent hydrogen bonds within the rod structure; and
the coil block is made up of:
radical homo- or copolymers derived from radical polymerization of at least one ethylene monomer of at least one of the following types: butadiene, (meth)acrylic, (meth)acrylamide, allyl, vinyl alcohol ester, and vinyl ether;
polycondensates of at least one of polyurethane, polyureas, aliphatic polyesters, aliphatic polyamides;
polymers obtained by opening cycles selected from polyesters and polyoxazolines
homopolymers of siloxane;
polymers obtained by metathesis;
copolymers comprising monomers obtained by cationic polymerization and
copolymers, salts and derivatives thereof.

36-37. (canceled)

38. A copolymer, configured as
a rod-block-coil di-block,
a coil-block-rod-block-coil tri-blocks, a rod-b-coil-b-rod tri-blocks, thereof.
